(19) United States
(12) Patent Application Publication
(10) Pub. No.: US 2024/0299729 A1
(43) Pub. Date: Sep. 12, 2024

(54) SURFACE MODIFICATION OF MEDICAL DEVICES

(71) Applicant: ABIOMED, Inc., Danvers, MA (US)

(72) Inventors: Vladimir Gilman, Danvers, MA (US); Scott Corbett, Danvers, MA (US)

(73) Assignee: ABIOMED, Inc., Danvers, MA (US)

(21) Appl. No.: 18/599,457

(22) Filed: Mar. 8, 2024

Related U.S. Application Data
(60) Provisional application No. 63/450,998, filed on Mar. 9, 2023.

(51) Int. Cl. A61M 60/13; A61M 60/226; A61M 60/411; A61M 60/50; A61M 60/818

(52) U.S. Cl. CPC A61M 60/13; A61M 60/226; A61M 60/411; A61M 60/50; A61M 60/818

(57) ABSTRACT

Techniques for delaying initiation of coagulation and suppressing fibrin formation may be provided. The disclosed techniques may include providing a percutaneous blood pump that may include a metal or ceramic surface (such as a surface of a shaft, bearing, rotor, stator, etc.) that has been modified with a bifunctional modifier. The modified surface may be within a motor section and/or pump section of the blood pump. The modified surface may be configured to interact with blood. When blood is allowed to interact with the modified surface, a desirable microenvironment may be formed. The bifunctional modifier may be a functionalized aminosilane, a functionalized aminosiloxane, and/or a functionalized silanetriol. The blood pump may be configured to have a purge fluid pass through at least a portion of the motor section and/or the pump section. The purge fluid may be free of anticoagulants.

### CROSS-REFERENCE TO RELATED APPLICATION

The present application claims priority to U.S. Provisional Patent Application No. 63/450,998, filed Mar. 9, 2023, the contents of which are incorporated by reference herein in its entirety.

### TECHNICAL FIELD

The present disclosure relates to medical devices, such as blood pumps (e.g., an intravascular blood pump, to support a blood flow in a patient's blood vessel and methods for purging such a pump in operation while inserted into a patient) having a blood-contacting surface.

### BACKGROUND

Blood pumps of different types are known, such as axial blood pumps, centrifugal blood pumps, or mixed-type blood pumps, where the blood flow is caused by both axial and radial forces. One example of a blood pump is the IMPELLA® line of blood pumps (e.g., IMPELLA 2.5® blood pump, IMPELLA CP® blood pump, IMPELLA 5.5® blood pump, etc.) which are products of Abiomed of Danvers, Mass. Intravascular blood pumps may be inserted into a patient's vessel such as the aorta by means of a catheter.

In some pump designs, a purge fluid may be deployed to keep blood from entering the mechanism and to mitigate the effects of blood on the pump mechanisms. The purge fluid may include aqueous dextrose solution (e.g., D5). In some instances, such a purge fluid also may include an anticoagulant such as heparin (typically the sodium salt of heparin), which is thought to keep the blood from coagulating in the gap between pump components such as an impeller shaft and the housing. In other instances, sodium bicarbonate has been introduced as an alternate to heparin.

### BRIEF SUMMARY

In various aspects, a percutaneous blood pump may be provided. The percutaneous blood pump may include a pumping device coupled to a catheter. The pumping device may include a motor section coupled to a pump section. The pump section may be configured to cause blood to flow from a blood inlet of the pumping device to a blood outlet of the pumping device.

A metal or ceramic surface of the pumping device may include a bifunctional modifier. The bifunctional modifier may be a functionalized aminosilane. The functionalized aminosilane may be 4-aminobutyltriethoxysilane. The bifunctional modifier may be a functionalized aminosiloxane. The bifunctional modifier may be a functionalized aminoalkyl silsequioxane. The functionalized aminosiloxane may be an aminoethylaminopropyl/methylsilsesquioxane, an aminopropyl/methylsilsesquioxane, an aminopropylsilsesquioxane, and/or an aminopropyl/vinylsilsesquioxane. The bifunctional modifier may be a functionalized silanetriol. The functionalized silanetriol may be a carboxyalkylsilanetriol. The carboxyalkylsilanetriol may include carboxyethylsilanetriol.

The bifunctional modifier may be selected to have (or form) a hydration layer with a thickness of no more than 1 nm from the metal or ceramic surface being modified. The bifunctional modifier may have a pH of 10-11. The bifunctional modifier may have a viscosity of 3-15 cSt. The bifunctional modifier may have a mole % of a functional group in the bifunctional modifier of 60-75%.

The metal or ceramic surface may be a surface of a shaft, bearing, rotor, or stator. The metal or ceramic surface may include an oxide. The metal or ceramic surface may include Cu, Fe, Al, Pb, Ti, Be, Ni, Si, Zr, Mn, Mo, Co, Bi, Zn, Mg, and/or Cr. The metal or ceramic surface may define a radial gap and/or axial gap in the motor section and/or the pump section.

The percutaneous blood pump may be configured to have a purge fluid pass through at least a portion of the motor section and/or the pump section (for example, through a radial gap or axial gap between two components of the motor section and/or pump section. The purge fluid may include an anticoagulant. The purge fluid may be free of anticoagulants.

In various aspects, a method for creating a microenvironment for delaying initiation of coagulation may be provided. The method may include providing a percutaneous blood pump as disclosed herein. The percutaneous blood pump may include a metal or ceramic surface that has been modified with a bifunctional modifier as disclosed herein. The metal or ceramic surface may be configured to contact blood. The method may include causing a purge fluid to pass through at least a portion of a motor section and/or a pump section of the percutaneous blood pump. The purge fluid may include an anticoagulant. The purge fluid may be free of anticoagulants.

### BRIEF DESCRIPTION OF DRAWINGS

Hereinafter, the invention will be explained by way of example with reference to the accompanying drawings. The accompanying drawings are not intended to be drawn to scale. In the drawings, each identical or nearly identical component that is illustrated in various figures is represented by a like numeral. For purposes of clarity, not every component may be labelled in every drawing.

FIG. 1 shows an illustration of a portion of a blood pump.

FIG. 2 shows an illustration of a portion of a blood pump, where a distal end is disposed within a right ventricle of a heart.

FIG. 3A is a schematic showing bonding a first modifier to a surface.

FIG. 3B is a schematic showing ionization in blood leading to localized alkalinization.

FIGS. 4A-4D are structures of bifunctional modifiers.

FIG. 4E is a schematic of a method for the anhydrous deposition of silanes.

FIG. 5A is a schematic of the ionization occurring using pH modifiers.

FIGS. 5B and 5C are depictions showing protection by NaHCO₃ at a pH<7 (5B) and pH>7 (5C).

FIG. 6 is an illustration of a portion of a blood pump.

FIG. 7 is an illustration of a system that includes a blood pump.

It should be understood that the appended drawings are not necessarily to scale, presenting a somewhat simplified representation of various features illustrative of the basic principles of the invention. The specific design features of the sequence of operations as disclosed herein, including, for example, specific dimensions, orientations, locations, and shapes of various illustrated components, will be determined in part by the particular intended application and use environment. Certain features of the illustrated embodiments have been enlarged or distorted relative to others to facilitate visualization and clear understanding. In particular, thin features may be thickened, for example, for clarity or illustration.

### DETAILED DESCRIPTION

As is known, blood pumps may be deployed in patients that require critical and life-saving care. Blood pumps also may be used to support high-risk procedures. In some pump designs, a purge fluid may be deployed to keep blood from entering the pump mechanism and to mitigate the effects of blood on the pump mechanisms. For example, the purge fluid may include an aqueous dextrose solution (e.g., D5), such as a 5% dextrose in water solution. In some instances, the purge fluid may also include an anticoagulant such as heparin (e.g., the sodium salt of heparin), which is thought to keep the blood from coagulating in the gap between pump components such as an impeller shaft and the housing. As will be appreciated, there may be instances in which heparin may not be suitable for all patients. In such instances, an alternative to heparin, such as sodium bicarbonate, has been appreciated by the inventors.

The inventors have recognized that a sodium bicarbonate purge fluid may provide environmental conditions that delay initiation of coagulation and suppress fibrin formation. For example, an overall decreased rate of clot formation where purge fluid and systemically anticoagulated blood mix in the axial gap. The inventors have also recognized that the protective effects of sodium bicarbonate may be attributed to reducing the probability of protein denaturation and platelet activation due to an increased pH (e.g., above 7.0, such as 7.8), and additional CO₂ in the purge fluid/blood mixtures. For example, certain advantageous microenvironments may be created.

As disclosed herein, the inventors have further recognized the benefit of pH-modifying (e.g., permanently pH-modifying) metal or ceramic surfaces to also achieve advantageous microenvironments for when those surfaces contact blood. For example, such microenvironment may delay initiation of coagulation and suppress fibrin formation, which may result in an overall decreased rate of clot formation. The microenvironment also may be configured to reduce the probability of protein denaturation and platelet activation due to an increased pH (e.g., above 7.0, such as 7.8) in blood mixtures in the microenvironment. For example, as shown herein, the inventors have recognized at high pH surface modification may provide a protective effect, such as to reduce protein denaturation and platelet activation, and thus, minimize clotting. The inventors have also recognized that such a surface modification may have an enzymatic effect on clot formation and clot lysis.

As will be appreciated, in embodiments having blood pumps with such surface modifications, the blood pumps may still be used with a suitable purge fluid (e.g., dextrose).

In some embodiments, surfaces may be modified via a bifunctional modifier. For example, in some embodiments, one function of the bifunctional modifier may serve to covalently bridge the modifier to the surface. As will be appreciated, in such instances, this may include a modifier that has an affinity. In some embodiments, the first modifier may be chosen based upon the surface being modified. For example, for some surfaces, such as ceramic surfaces, the first modifier may include an alkoxy functional group, such as a methoxy functional group (—OCH₃). For epoxy surfaces, the first modifier may include a carbonide or an epoxide. A hydroxyl modifier may be used for ceramics and metals. A carboxyl group also could be used as a modifier.

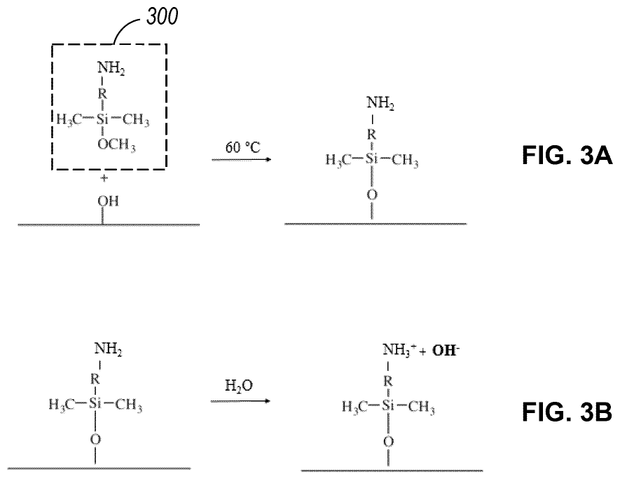

An example of a first modifier (300) bonding to a surface is illustrated in FIG. 3A. In FIG. 3A, a solution of isopropanol at 60 degrees Celsius is utilized to facilitate the bonding of the modifier to the surface. As will be appreciated, bonding also may be facilitated using other alcohol solutions at other suitable temperatures (e.g., between 20 degrees Celsius and 120 degrees Celsius).

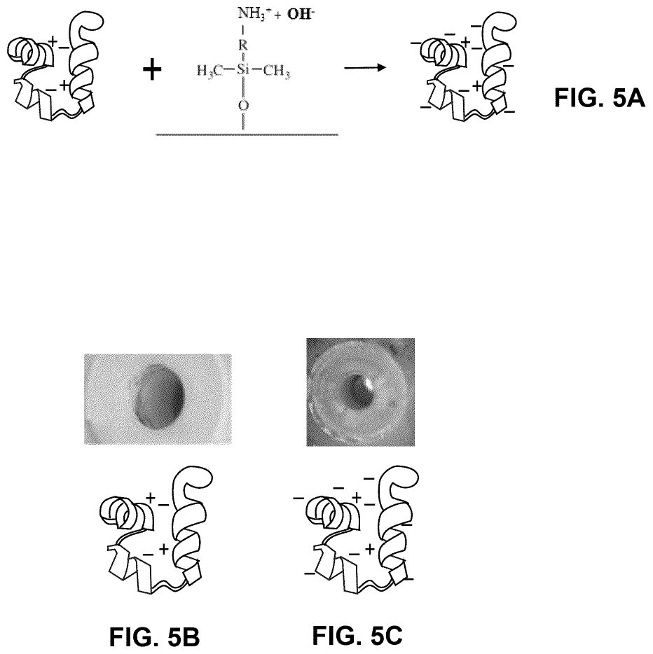

In some embodiments, the first modifier may be configured as a pH adjuster. As shown in FIGS. 3A and 3B, for example, the pH adjuster may include NH₂. The ionization that may result in blood is also shown in FIG. 3B (in which NH₂ is converted to NH₃⁺) and FIGS. 5A-5C In FIG. 3B, the putative ionization of the modifier in blood (left side of FIG. 3B) via hydrolysis may lead to localized alkalinization (right side of FIG. 3B) In some embodiments, through the surface modification, the pH may reduce denaturation because it may keep the negative charge on the protein molecule. As shown in these figures, the OH group may keep the negative charge on the protein. In FIG. 5A, the left side shows charges on the molecule at low pH (<7, e.g., D5 alone), while the right side shows charges at higher pHs (>7, e.g., D5 and blood). FIGS. 5B and 5C show images of protection by NaHCO₃, at lower pHs (5B, pH<7) and higher pHs (5C, pH>7).

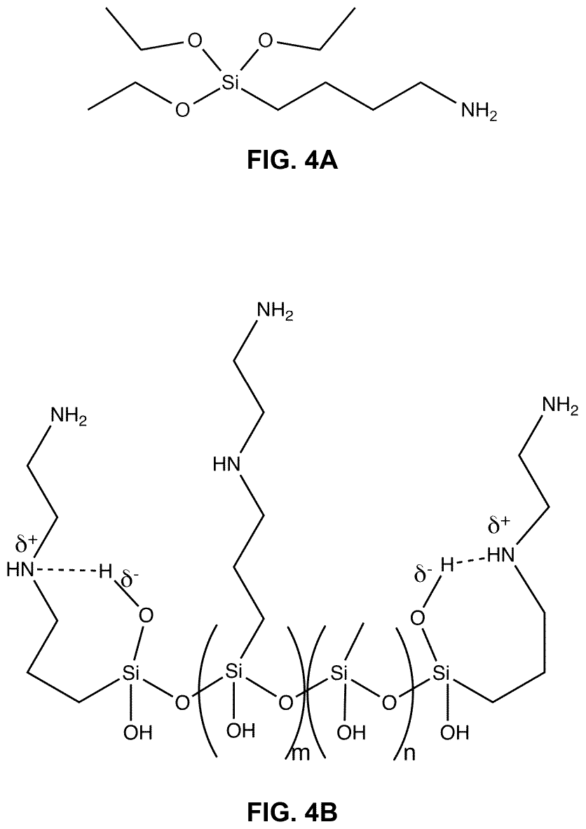

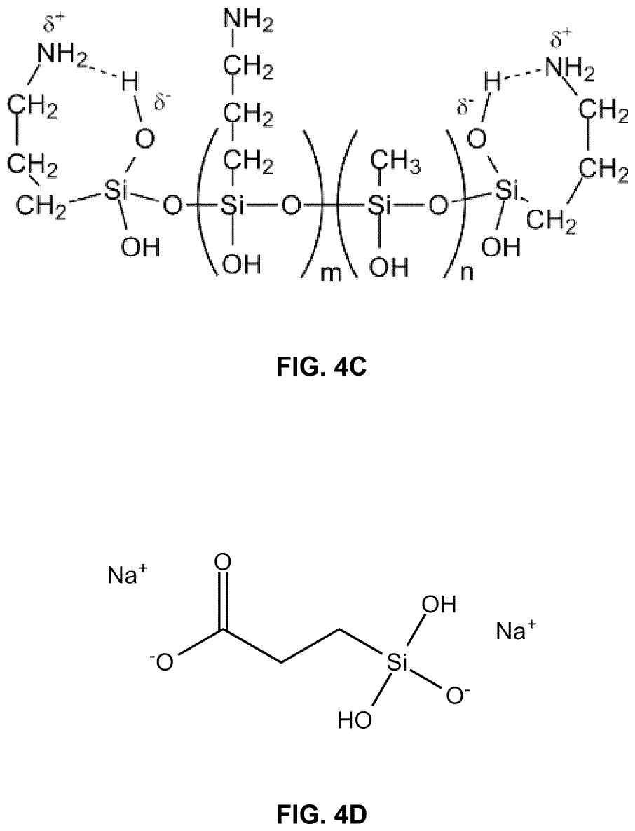

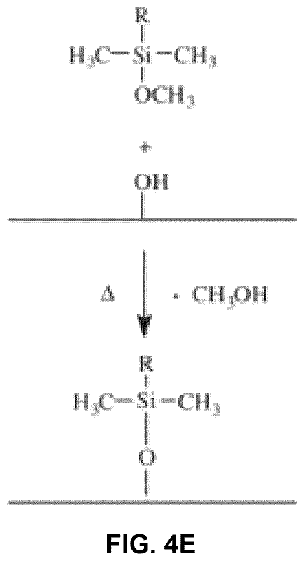

In some embodiments, the bifunctional modifiers may be a functionalized aminosilane. In some embodiments, the functionalized aminosilane may be a functionalized triethoxysilane, having the formula (EtO)₃Si(CH₂)ₙNH₂ where n=3-11. A non-limiting example of such an aminosilane is shown in FIG. 4A: 4-aminobutyltriethoxysilane. Other non-limiting examples include 3-aminopropyltriethoxysilane, 5-aminopentyltricthoxysilane, 6-aminohexyltriethoxysilane, and 11-aminoundecyltriethoxysilane. In some embodiments, the functionalized aminosilane may be a functionalized silane, having the formula R1R2R3Si(CH₂)ₙNH₂ where n=3-11, and R1, R2, R3 are independently an alkyl or alkoxy, where at least two of R1, R2, and R3 are alkoxy groups. The alkyl and alkoxy groups may be carbon chain lengths of 1-5. Non-limiting examples of such include 3-Aminopropyl(diethoxy)methylsilane, and 4-aminobutyl(diethoxy)ethylsilane. The bifunctional modifiers also may be a functionalized aminosiloxane. The bifunctional modifiers also may be a functionalized aminoalkyl silsequioxane. Non-limiting examples of such bifunctional modifiers are shown in FIGS. 4B-4C, and include an aminoethylaminopropyl/methylsilsesquioxane (FIG. 4B); and/or an aminopropyl/methylsilsesquioxane (FIG. 4C). Other functionalized aminosiloxanes may include an aminopropylsilsesquioxane and/or an aminopropyl/vinylsilsesquioxane. The bifunctional modifier may be a functionalized silanetriol, such as a carboxyalkylsilanetriol, such as carboxyethylsilanetriol, and may be a salt of the silantriol, such as carboxyethylsilanetriol, disodium salt (FIG. 4D). An example showing the anhydrous deposition of silanes is schematically represented in FIG. 4E.

In some embodiments, the bifunctional modifiers may be selected to have a high hydration layer. In some embodiments, the bifunctional modifiers may be selected to have a hydration layer with a thickness of no more than 1 nm from the underlying surface being modified. For example, the hydration layer may have a thickness of 0.1 nm-1 nm. The hydration layer may have a thickness of at least 0.1 nm, at least 0.2 nm, at least 0.3 nm, at least 0.4 nm, or at least 0.5 nm. The hydration layer may have a thickness of no more than 0.3 nm, no more than 0.4 nm, no more than 0.5 nm, no more than 0.6 nm, no more than 0.8 nm, or no more than 1.0 nm. As will be appreciated, the hydration layer may have other suitable thicknesses in other embodiments. In some embodiments, such hydration layers may prevent or impede molecules (such as molecules in blood) from reaching the surface.

In some embodiments, the bifunctional modifier may have a pH of 10-11. In some embodiments, the bifunctional modifier may have a viscosity of 3-15 cSt. In some embodiments, the mole % of the functional group in the bifunctional modifier molecule may be 60-75%. In some embodiments, the bifunctional modifier may be provided in an aqueous solution.

Although disclosed herein as having benefits to a high pH surface modification, it will be appreciated that benefits may be achieved by having a low pH surface modification in some instances. In such instances, an appropriate bifunctional modifier may be chosen to create such an environment.

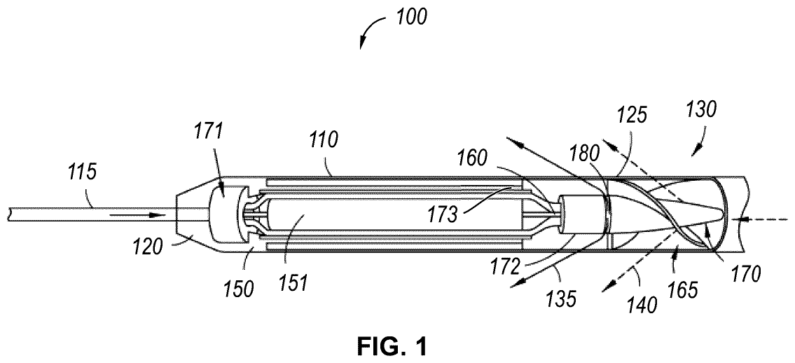

Turning now to FIG. 1, a pump (100) is shown having a tubular member with a drive section (110) and a pump section (130), a catheter 115 attached to a proximal end (120) of the drive section (110) (e.g., the end of the drive section closer to the doctor or “rear end” of the drive section) and with lines extending therethrough for the power supply to the drive section (110), and the pump section (130) fastened at the distal end (125) of the drive section. The drive section (110) may include a motor housing (150) having an electric motor (151) disposed therein, with the motor shaft (160) of the electric motor distally protruding out of the drive section (110) and into the pump section (130). The pump section (130) in turn may include a pump housing (165) (which may be a tubular pump housing) having an impeller (170) rotating therein which is seated on the end of the motor shaft (160) protruding out of the motor housing (150). The motor shaft (160) may be mounted in the motor housing in two bearings (171, 172) which are maximally removed from each other in order to guarantee a true, exactly centered guidance of the impeller (170) within the motor housing (150). Different bearing types may be used in different pump designs. As illustrated in FIG. 1, bearing (171) may include a radial ball bearing and bearing (172) may include an axial-radial sliding bearing. As illustrated in FIG. 1, blood (140) may exit the outflow cage of the pump housing (165). Blood that would otherwise enter into the motor housing (150) may be furthermore counteracted by a purge fluid (135) being passed through the motor housing and the impeller-side shaft seal bearing. Accordingly, the purge fluid may pass through the gap of the impeller-side radial sliding bearing so as to prevent blood from entering into the housing. This may be done by having a purge fluid pressure that is higher than the pressure present in the blood.

As illustrated in FIG. 1, the purge fluid (135) may fill the motor housing (150) of the pump to form a lubricating film in the bearings (171, 172) of the pump. As described in U.S. Patent Publication No. 2015/0051436, for example, the purge fluid (135) may form a lubricating film in a bearing gap (180) of the axial slide bearing of a pump. Purge fluids are described as being fed through a purge-fluid feed line and flowing through the radial bearing gap (173) located at the distal end of the motor housing (150) and then also flowing through the bearing gap (180) of the axial sliding bearing. The purge fluids fed in this manner may be responsible for hemo-dilution and reduce blood retention time under the impeller (170).

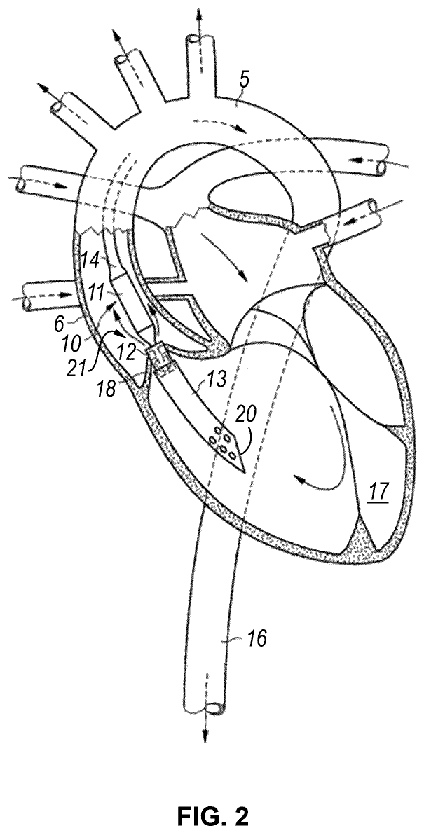

FIG. 2 illustrates the employment of a blood pump for supporting, in this particular example, the left ventricle. As shown in this figure, the blood pump may include a catheter (14) and a pumping device (10) attached to the catheter (14). The pumping device (10) may include a motor section (11) and a pump section (12) which are disposed coaxially one behind the other and result in a rod-shaped construction form. The pump section (12) may include an extension in the form of a flexible suction hose (13), a tubular member often referred to as “cannula.” An impeller may be provided in the pump section (12) to cause blood flow from a blood flow inlet (20), through a portion of the pumping device, such as through a portion of the motor section and/or the pump section), to a blood flow outlet (21), and rotation of the impeller is caused by an electric motor disposed in the motor section (11). The blood pump may be placed such that it lies primarily in the ascending aorta (6) leading to the aortic arch (5). The aortic valve (18) comes to lie, in the closed state, against the outer side of the pump section 12 or its suction hose (13) that lies substantially in the left ventricle (17). The blood pump with the suction hose (13) in front may be advanced into the represented position by advancing the catheter (14), optionally employing a guide wire. In so doing, the suction hose (13) may pass the aortic valve (18) retrograde, so the blood is sucked in through the suction hose (13) and pumped into the aorta (16).

As will be appreciated, the use of the blood pump need not be restricted to the application represented in FIG. 2, which merely involves a typical example of application. Thus, the pump can also be inserted through other peripheral vessels, such as the subclavian artery. Alternatively, reverse applications for the right ventricle may be envisioned. As will be further appreciated, other suitable pump arrangements may be used in other embodiments.

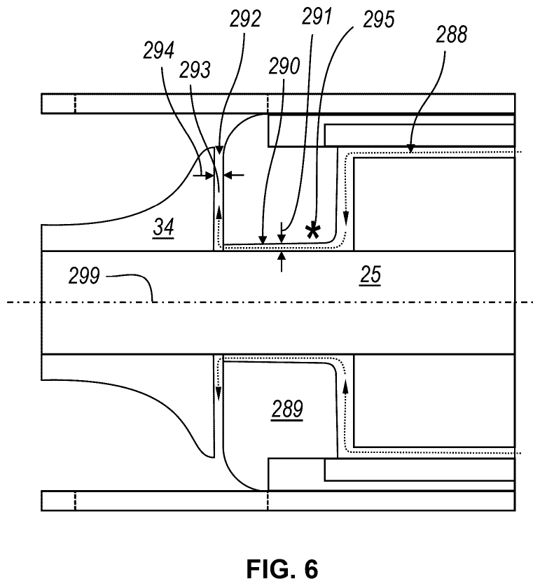

FIG. 6 illustrates an embodiment showing fluid flowing through purge gaps. As shown in this view, the pump may include first gap (290) and second gap (292). In some embodiments, the first gap (290) may include a small radial gap (291) between an outer surface of a shaft (25) and an inner surface of a sleeve bearing (289), which may control the purge flow rate as the purge fluid (288) flows through the purge gaps. As will be appreciated, metal or ceramic surfaces, such as the bearing surfaces around these gaps, may be surface modified (see surfaces near “*” (295) in FIG. 6). As will be appreciated, in other embodiment, the shafts also may be modified as described herein. In some embodiments, both the shafts and bearings are surface modified. As will be further appreciated, the surface need not have the same modifier, although it may be the same in some embodiments.

As will also be understood, in some embodiments, any metal or ceramic surface of the blood pump may be modified. In some embodiments, the metal surfaces may be surfaces that contact blood, similar to blood contact bearings and shafts described herein. The metal surface may be any appropriate metal. The metal may comprise an oxide. For example, the metal may include, Cu, Fe, Al, Pb, Ti, Be, Ni, Si, Zr, Mn, Mo, Co, Bi, Zn, Mg, and/or Cr, although other metallic materials may be used. Such surfaces may be readily modified as described herein, or via other suitable methods (e.g., silanization).

In some embodiments, the small radial gap (291) may extend in a radial direction between 4 μm and 9 μm, such as between 5 and 6 μm for some pumps and between 7 and 8 μm for other pumps. In some embodiments, the first gap may largely contain purge fluid. In some embodiments, due to flow co-mixing, some blood components may potentially reach the distal end of this gap. Due to the small dimensions, heat, and high shear, the biological material buildup in this location may include a denatured protein, which may result in rising purge pressures and increased friction leading to high motor current. In such embodiments, by modifying one more of the surfaces, a microenvironment may be configured to reduce denaturation and adsorption of blood proteins in the gap. In some embodiments, a purge fluid may be introduced. The purge fluid may be, e.g., a saline purge fluid. The purge fluid may include one or more anticoagulants, such as heparin or sodium bicarbonate. In some embodiments, the purge fluid may be free of anticoagulants. Although described as having a purge fluid, it will be appreciated that a purge fluid need not be used in all medical devices, although one or more surface may be modified to create the disclosed microenvironments. For example, in some embodiments, the blood pump may be configured to be purgeless (e.g., with just blood flowing therethrough), with the surface modification serving to create the microenvironments, such as to reduce coagulation.

In one embodiment, the surface modification may prevent the agglomeration of the protein by increasing the electrostatic charge of the serum protein, and therefore reduces formation of bio-deposition.

In some embodiments, the second gap (292) may include an axial gap (293) between the impeller (34) and a sleeve bearing (289). In some embodiments, the axial gap (293) may extend in an axial direction (e.g., a distance (294) parallel to axis (299) of the impeller shaft) of between about 90 and 110 μm, such as about 100 μm.

In some embodiments, the second gap may be where purge fluid (if used) and blood may mix. Purge fluid may flow along towards the impeller, while system blood is pulled into the gap setting up a clockwise flow pattern in this micro-environment.

Purge flow rates are typically in the range of about 2 mL/hour to about 30 mL/hour. This results in a purge pressure of about 1100 mmHg to about 300 mmHg. Typical purge flows for some blood pumps are about 5 mL/hour to about 20 mL/hour, although specific models may have purge flows from about 2 mL/hour to about 10 mL/hour.

For surgical patients, surgeons prefer not to administer heparin in the first few days after surgery. For these patients, then, purge fluids that contain no heparin are preferred.

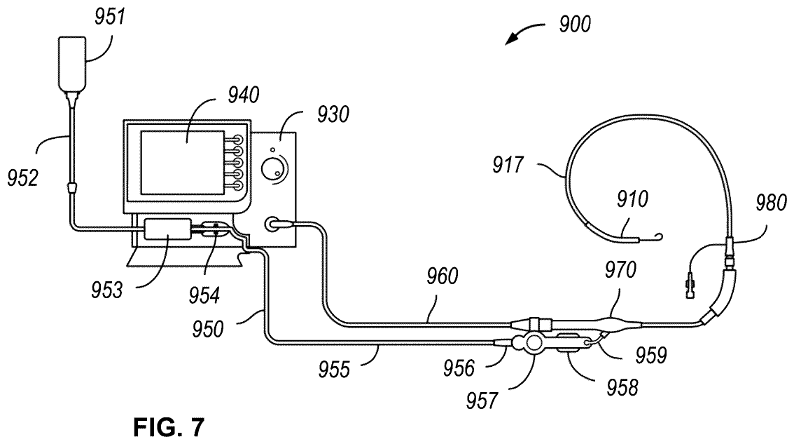

Referring to FIG. 7, a blood pump assembly (900) may include a blood pump (910) fluidically connected to a container (951) (such as a purge bag) that contains a purging fluid as disclosed herein, through a purging device (953). The blood pump assembly (900) also may include a controller (930) (e.g., an AUTOMATED IMPELLA CONTROLLER® (AIC) blood pump controller from Abiomed, Inc., Danvers, MA), a display (940), a connector cable (960), a plug (970), and a repositioning unit (980). As shown, the controller (930) may include a display (940). Controller (930) may monitor and controls blood pump (910). During operation, purging device (953) may deliver a purge fluid as disclosed herein to blood pump (910) through a first line (950, 955) (e.g., a tube), through one or more components (956, 957, 958, 959) and through a catheter tube (917), such as to prevent blood from entering the motor (not shown) within a motor housing of the pump. Connector cable (960) may provide an electrical connection between blood pump (910) and controller (930). Plug (970) connects catheter tube (917), purging device (953), and connector cable (960). In some embodiments, plug (970) may include a memory for storing operating parameters in case the patient needs to be transferred to another controller. Repositioning unit (980) may be used to reposition blood pump (910). As shown in this view, the fluid line (950, 955) may be separate from the connector cable (960) having one or more electrical wires.

One or more of the metal or ceramic surfaces within the blood pump, such as one or more ceramic and/or metallic surfaces that may contact blood (e.g., one or more surfaces of a shaft, a bearing, a rotor, a stator, etc.) may be modified with a bifunctional modifier as disclosed herein.

In some embodiments, a method may include operating the pump, which may include rotating the impeller of the pump. In that regard, the method may involve pumping a flow of blood from the blood flow inlet, through a portion of the pumping device, such as through a portion of the motor section and/or the pump section, and out a blood flow exit. In such embodiment, the blood may pass through one or more of the modified surfaces (e.g., through the axial gaps disclosed herein). As described herein, such modified surfaces may create microenvironments that may minimize and/or prevent agglomeration of protein as blood passes therethrough.

In some embodiments, the method may also include flowing the purge fluid into the disclosed gaps of the pump. This may be done by controlling the purging device (953) (which may include, e.g., a positive displacement pump). This may include flowing the purge fluid into a blood pump as disclosed herein, to allow the purge fluid to mix with blood in the blood pump. This may include flowing the purge fluid through a first gap between a bearing and an outer surface of a rotatable shaft coupled to the impeller, the bearing and gap being disposed within a lumen of a tubular member. In some embodiments, this may include flowing the fluid through a second gap after passing through the first gap, the second gap being between the bearing and a surface of the impeller facing the bearing. In such embodiments, the blood, and optionally a blood/purge fluid mixture, may contact the surface-modified metal or ceramic surface as disclosed herein to create the disclosed, desired microenvironment.

In some embodiments, the controller may be configured to control the flow rate of the purge fluid into the pump based on a speed of the impeller. For example, at low impeller rotational velocities, the flow rate of the purge fluid may be relatively low, and as the impeller increases in speed, the flow rate of the fluid will automatically be increased to offset the increased pressure of blood attempting to enter, e.g., the gap(s) of the pump.

In various aspects, a method for creating a microenvironment for delaying initiation of coagulation may be provided. The method may include providing a percutaneous blood pump comprising a metal or ceramic surface that has been modified with a bifunctional modifier as disclosed herein. This may include operating the blood pump in such a manner that blood flowing through the blood pump interacts with the bifunctional modifier on the surface.

Although embodiments disclosed herein include affecting the micro-environment of purges gaps (e.g., axial and/or radial gaps) within a blood pump (e.g., a percutaneous blood pump), such surface modifications may be used on blood-contacting surfaces and on other medical devices. For example, in some embodiments, the high pH surface modification could be used on metal cables or cables having metal and/or ceramic components that pass through a patient's skin and/or through areas of the patient's vasculature. It may be used on blood contact surfaces for pacemakers. It also may be used to coat batteries and/or controllers that may be exposed to blood and/or may be susceptible to bacteria. As will be appreciated, in such instances, as with the above, wash out of the surface may be performed as well, such as to encourage microbes to not attached to surfaces and create a biofilm.

While particular embodiments of this technology have been described, it will be evident to those skilled in the art that the present technology may be embodied in other specific forms without departing from the essential characteristics thereof. The present embodiments and examples are therefore to be considered in all respects as illustrative and not restrictive. It will further be understood that any reference herein to subject matter known in the field does not, unless the contrary indication appears, constitute an admission that such subject matter is commonly known by those skilled in the art to which the present technology relates.

1. A percutaneous blood pump comprising:
a pumping device coupled to a catheter, the pumping device comprising a motor section coupled to a pump section, the pump section configured to cause blood to flow from a blood inlet of the pumping device to a blood outlet of the pumping device,
wherein a metal or ceramic surface of the pumping device comprises a bifunctional modifier.

2. The percutaneous blood pump of claim 1, wherein the metal or ceramic surface is a surface of a shaft, bearing, rotor, or stator.

3. The percutaneous blood pump of claim 1, wherein the metal or ceramic surface comprises an oxide.

4. The percutaneous blood pump of claim 1, wherein the metal or ceramic surface comprises Cu, Fe, Al, Pb, Ti, Be, Ni, Si, Zr, Mn, Mo, Co, Bi, Zn, Mg, and/or Cr.

5. The percutaneous blood pump of claim 1, wherein at least one metal or ceramic surface defines a radial gap and/or axial gap in the motor section and/or the pump section.

6. The percutaneous blood pump of claim 1, wherein the percutaneous blood pump is configured to have a purge fluid pass through at least a portion of the motor section and/or the pump section.

7. The percutaneous blood pump of claim 6, wherein the purge fluid is free of anticoagulants.

8. The percutaneous blood pump of claim 1, wherein the bifunctional modifier is a functionalized aminosilane.

9. The percutaneous blood pump of claim 8, wherein the functionalized aminosilane is 4-aminobutyltriethoxysilane.

10. The percutaneous blood pump of claim 1, wherein the bifunctional modifier is a functionalized aminosiloxane.

11. The percutaneous blood pump of claim 10, wherein the bifunctional modifier is a functionalized aminoalkyl silsequioxane.

12. The percutaneous blood pump of claim 10, wherein the functionalized aminosiloxane is an aminoethylaminopropyl/methylsilsesquioxane, an aminopropyl/methylsilsesquioxane, an aminopropylsilsesquioxane, and/or an aminopropyl/vinylsilsesquioxane.

13. The percutaneous blood pump of claim 1, wherein the bifunctional modifier is a functionalized silanetriol.

14. The percutaneous blood pump of claim 13, wherein the functionalized silanetriol is a carboxyalkylsilanetriol.

15. The percutaneous blood pump of claim 14, wherein the carboxyalkylsilanetriol comprises carboxyethylsilanetriol.

16. The percutaneous blood pump of claim 1, wherein the bifunctional modifier is selected to have a hydration layer with a thickness of no more than 1 nm from the metal or ceramic surface being modified.

17. The percutaneous blood pump of claim 1, wherein the bifunctional modifier has a pH of 10-11.

18. The percutaneous blood pump of claim 1, wherein the bifunctional modifier has a viscosity of 3-15 cSt.

19. The percutaneous blood pump of claim 1, wherein the bifunctional modifier has a mole % of a functional group in the bifunctional modifier of 60-75%.

20. A method for creating a microenvironment for delaying initiation of coagulation, comprising:
providing a percutaneous blood pump comprising a metal or ceramic surface that has been modified with a bifunctional modifier.

21-39. (canceled)
